(19) United States
(12) Patent Application Publication
(10) Pub. No.: US 2022/0184326 A1
(43) Pub. Date: Jun. 16, 2022

(54) MEDICINAL NEBULIZER AND METHOD OF DISPENSING MEDICAMENT

(71) Applicant: Health Research, Inc., Buffalo, NY (US)

(72) Inventor: Khurshid GURU, East Amherst, NY (US)

(21) Appl. No.: 17/438,620

(22) PCT Filed: Mar. 11, 2020
(86) PCT No.: PCT/US2020/022204

§ 371 Date: Sep. 13, 2021

Related U.S. Application Data
(60) Provisional application No. 62/816,822, filed on Mar. 11, 2019.

(51) Int. Cl. A61M 11/02

(52) U.S. Cl. CPC A61M 11/02; A61M 2205/071

(57) ABSTRACT

A nebulizer for medicament is provided. The nebulizer includes a cup having an interior surface. An axle is attached to the cup at least at a location along a central axis of the cup such that rotation of the axle causes a rotation of the cup about the central axis. A fan is operably attached to the axle and configured to generate a flow of air adjacent to the cup. A drum is disposed around a circumference of the cup, and configured to contain the generated flow of air near the cup. A conduit is configured to receive liquid medicament and deposit such medicament on a surface of the cup.

### CROSS-REFERENCE TO RELATED APPLICATIONS

This application claims priority to U.S. Provisional Application No. 62/816,822, filed on Mar. 11, 2019, now pending, the disclosure of which is incorporated herein by reference.

### FIELD OF THE DISCLOSURE

The present disclosure relates to medicinal nebulizer.

### BACKGROUND OF THE DISCLOSURE

Nebulizers and inhalers are commonly used by individuals needing medications delivered into the body via the lungs. Such medications may treat conditions like asthma or chronic obstructive pulmonary disease (COPD). However, the use of a nebulizer often involves an electrically-powered air pump/compressor in order to deliver a flow of air through a tube to an attachment which atomizes the liquid medicament into the flow of air and provides the aerosol to the patient. However, such nebulizers have several disadvantages including, the cost of the air pump, the need for electrical power in order to use the system, and the noise inherent in using an air pump. Even though the air pump is reused and thereby only incurring the initial expense of this component, this large upfront cost may be insurmountable for a significant number of people—especially those living in developing countries. Furthermore, the same populations of people may not have ready access to the electrical power required to power such traditional nebulizers. Accordingly, there remains a need for a nebulizer that is inexpensive to make and inexpensive to use.

### BRIEF SUMMARY OF THE DISCLOSURE

In one aspect, the present disclosure provides a nebulizer for medicament. The nebulizer has a cup with an interior surface. An axle is attached to the cup at least at a location along a central axis of the cup such that rotation of the axle about a longitudinal axis causes a rotation of the cup about the central axis. A fan is operably attached to the axle and configured to generate a flow of air adjacent to the cup. A drum is disposed around a circumference of the cup. The drum is configured to contain the generated flow of air near the cup. The nebulizer includes a mover for rotating the axle. A conduit is configured so as to transport medicament from the vessel to the interior surface of the cup. In some embodiments, a vessel for containing a liquid medicament is provided. In such an embodiment, the conduit may be configured between (i.e., fluidically between) the vessel and the cup.

### DESCRIPTION OF THE DRAWINGS

For a fuller understanding of the nature and objects of the disclosure, reference should be made to the following detailed description taken in conjunction with the accompanying drawings, in which:

FIG. 1 is a perspective view diagram of a nebulizer according to an embodiment of the present disclosure;

FIG. 2 is another perspective view of the nebulizer of FIG. 1, wherein a portion is shown as transparent to better illustrate internal components;

FIG. 3 is a side-elevation view of the nebulizer of FIGS. 1-2, wherein portions are shown as transparent to better illustrate internal components;

FIG. 4 is a front-elevation view of the nebulizer of FIGS. 1-3, wherein portions are shown as transparent to better illustrate internal components;

FIG. 5 is a view of several internal components of the nebulizer of FIGS. 1-4;

FIG. 6 is another perspective view of the nebulizer of FIGS. 1-4;

FIG. 7 is an exploded view of portions of the nebulizer of FIGS. 1-4;

FIG. 8 is another perspective view of the nebulizer of FIGS. 1-4;

FIG. 9 is another perspective view of the nebulizer of FIGS. 1-4, wherein portions are shown as transparent to better illustrate internal components; and

FIG. 10 is a top plan view of the nebulizer of FIGS. 1-4, wherein portions are shown as transparent to better illustrate internal components.

### DETAILED DESCRIPTION OF THE DISCLOSURE

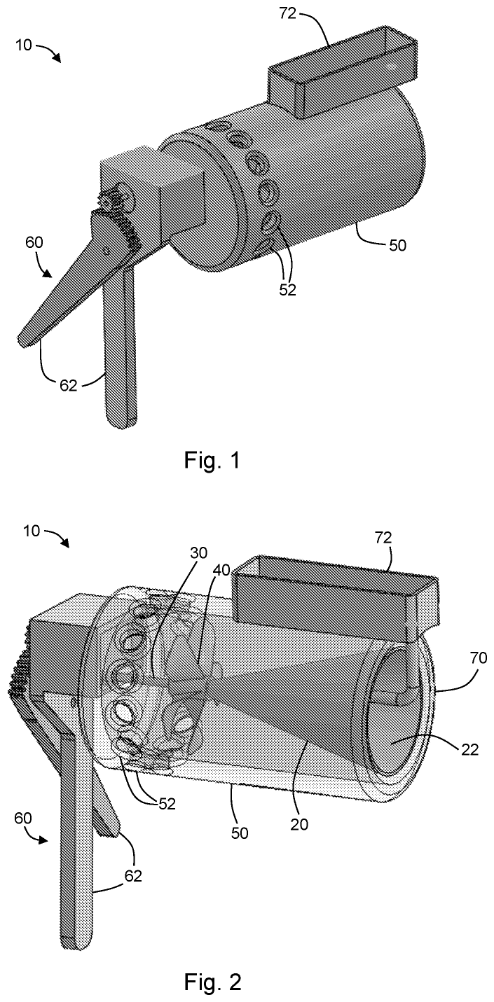

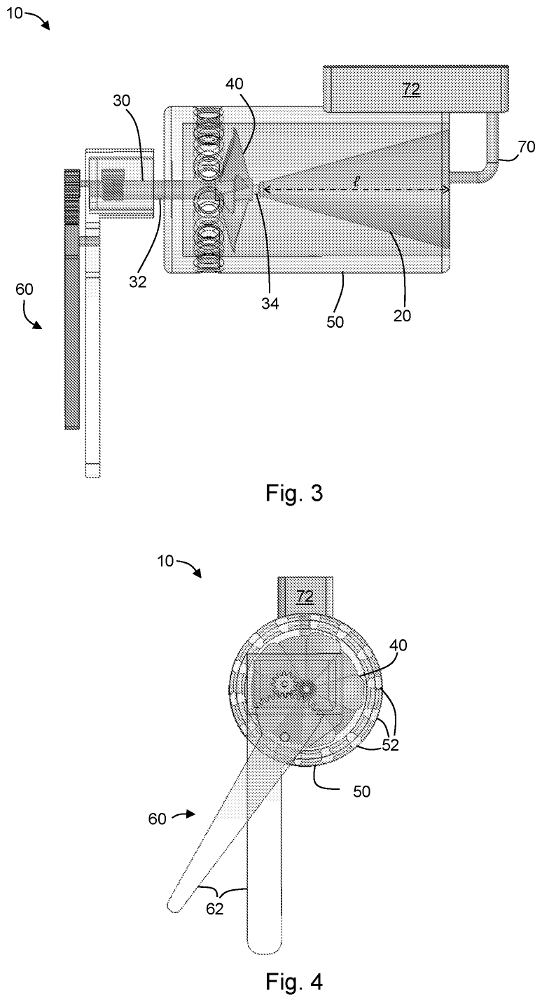

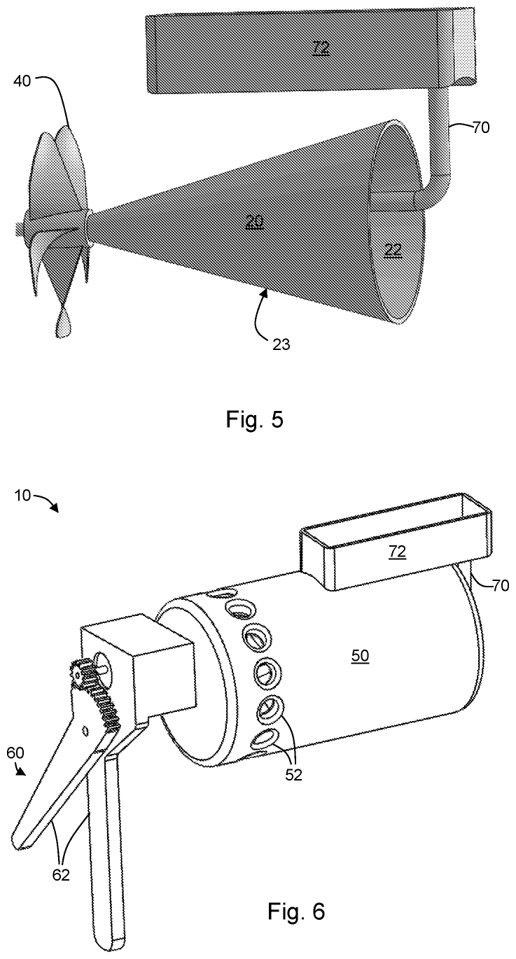

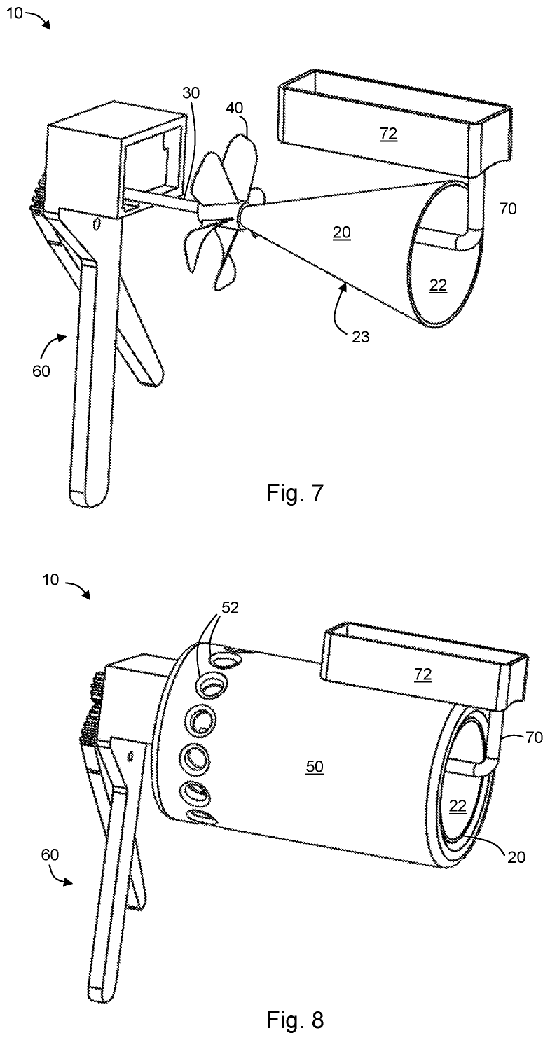

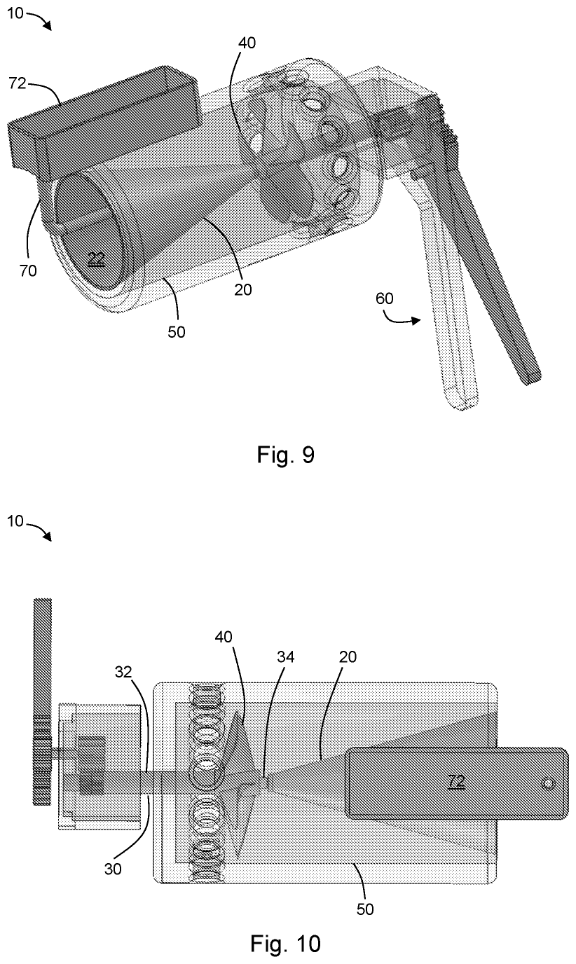

With reference to FIGS. 1-10, the present disclosure may be embodied as a nebulizer 10 for a medicament. The nebulizer 10 includes a cup 20 having an interior surface 22 and an exterior surface 23. The cup may be configured in any shape. In some embodiments, the cup 20 is advantageously shaped to be conical (i.e., cone-shaped) as further described herein. The cup may have one or more open ends. For example, the cup 20 depicted in the figures has a first open end at a large end of the cone shape. In other embodiments of such a conical cup, the cup may be open on both ends (i.e., a large diameter end and a small diameter end). The cup 20 may be made from various materials. In some embodiments, the cup is made from one or more air permeable materials, for example, paper, fabric, etc. In some embodiments, the cup is made from a mesh material. The term “mesh” should be interpreted broadly and may include woven materials, solid materials (such as, for example, metal foils) with a plurality of holes, or other configurations in which a material is physically structured to be permeable by air. In some embodiments, the interior surface of the cup is smooth. In some embodiments, the interior surface of the cup is not smooth. For example, the interior surface of the cup may have protrusions or other enhancements that may be advantageous as further described below.

An axle 30 is attached to the cup 20 at a location along a central axis of the cup 20 such that rotation of the axle 30 (i.e., about a longitudinal axis of the axle 30 as is a common use of such axles) will cause a rotation of the cup 20 about its central axis. The nebulizer 10 further include a fan 40 operably attached to the axle 30. The fan 40 is configured to generate a flow of air adjacent to the cup 20. In some embodiments, the axle 30 comprises a hollow outer shaft 32 and an inner shaft 34 coaxially disposed within the hollow outer shaft 32. In this way, the hollow outer shaft 32 may be driven at a rotational speed which is different than the inner shaft 34 (though the shafts may instead be driven at the same speed). In some embodiments of such an axle, the fan 40 may be attached to the hollow outer shaft 32 and the cup 20 may be attached to the inner shaft 34. In this way, the fan 40 may be driven at a speed which is faster than, slower than, or the same as the speed of the cup 20. The nebulizer may further include a gearbox. Such a gearbox may be used, for example, to accomplish the differing rotation speeds of the outer shaft and inner shaft.

A drum 50 is disposed around a circumference of the cup 20. The drum 50 is configured to contain the generated flow of air generated by the fan 40 and maintain the flow near the cup 20. By near, it is intended that the flow of air be sufficiently close to the cup such that liquid on the cup (e.g., on a surface of the cup) may be taken up by the flow and expelled from the nebulizer. For example, in the embodiment shown in the figures, the drum 50 is configured such that air generated by the fan 40 will flow along the length of the cone-shaped cup 20 (length l along the longitudinal axis) and exit the nebulizer 10 at the large, open end of the cup 20. In some embodiments, the drum 50 may include one or more ports 52 so as to allow ambient air to enter the nebulizer 10 and be propelled by the fan 40. The port(s) may be configured at an end of the drum. For example, the port(s) may be on a side of the drum near an end (as depicted in the figures) and/or on the end of the drum or otherwise. The drum may be shaped similar to the shape of the cup or similar to the shape of a portion of the cup. In this way, the flow of air propelled by the fan may further be guided along at least a portion of the surface of the cup.

The nebulizer 10 may further include a mover 60 for rotating the axle 30. The mover 60 may be, for example, a pair of handles 62 such as the handles 62 depicted in the figures. Such a configuration using a pair of handles 62 may be configured to rotate the axle 30 when the handles 62 are squeezed together by a user. For example, one handle 62 may be stationary, while the other is movable. Other suitable embodiments of movers will be apparent to one having skill in the art in light of the present disclosure. For example, the mover may be a pull string, push button, crank, slide, etc. In some embodiments, the axle may be rotated by an energy store such as, for example, a mechanical energy store (e.g., a spring, etc.) and the mover may be configured to engage the energy store. For example, the mover may wind a spring (e.g., store energy in a spring) and the stored energy may be released to rotate the axle. In some embodiments, the stored energy may be released without user intervention. In some embodiments, an actuator may be used to trigger release of the stored energy.

A conduit 70 is provided and configured to receive medicament and deposit received medicament on a surface of the cup. For example, the conduit may be configured to deposit medicament on the interior surface 22 of the cup 20 (when a liquid medicament is introduced into the conduit 70). The conduit may be of any length or configuration suitable to a particular design. For example, the conduit may be a short length which is essentially a through-hole in the drum. In some embodiments, the nebulizer 10 includes a vessel 72 for containing medicament. The vessel 72 is configured to cooperate with the conduit 70 so as to dispense the medicament to the cup 20 from the vessel 72 via the conduit 70. In other embodiments, the vessel is a removable component and may not make up a portion of the nebulizer itself. For example, the vessel may be a disposable component which can be attached to dispense medicament and disposed of once the medicament has been dispensed. The conduit 70 and/or the vessel 72 may include a valve for controlling the flow of medicament. For example, the valve may be configured such that medicament flows (i.e., is dispensed to the cup) only when the cup is rotated. In some embodiments, the conduit 70 and/or the vessel 72 is configured such that the medicament is dispensed at a metered (i.e., controlled) rate. In some embodiments, the metered rate is adjustable.

The principle of operation of embodiments of the presently-disclosed nebulizer 10 is such that medicament is dispensed on the interior surface 22 of the cup 20 such that the medicament may be carried off by a flow of air generated by the fan 40. The rotation of the cup 20 is intended at least in part to provide for a better distribution of medicament onto the interior surface of the cup 20. In this way, the medicament is more likely to be carried from the nebulizer as a fine mist (i.e., small droplets) rather than a spray of larger droplets. Other enhancements may provide further improvement in the efficiency of generating the aerosol and/or in the characteristics of the generated aerosol. For example, where the cup 20 is made from an air-permeable material, such as a mesh material, the medicament may be able to aerosolize over the surface of the cup 20 rather than only at an edge of the cup 20. In another example, a cone-shaped cup may provide improved distribution of the dispensed medicament by, for example, using centrifugal force to move the medicament along the length of the cup (i.e., towards the open end of the cone-shaped cup). Enhancements on the interior surface 22 of the cup 20 may provide further improvements in the distribution of the medicament and/or in the ability of the liquid medicament to be taken up by the flow of air.

### ADDITIONAL EXAMPLES

Example 1. A nebulizer for medicament, comprising a cup having an interior surface; an axle attached to the cup at least at a location along a central axis of the cup such that rotation of the axle causes a rotation of the cup about the central axis; a fan operably attached to the axle and configured to generate a flow of air adjacent to the cup; a drum disposed around a circumference of the cup, and configured to contain the generated flow of air near the cup; and a conduit configured to receive liquid medicament and deposit such medicament on a surface of the cup.

Example 2. The nebulizer of example 1, further comprising a vessel for containing a liquid medicament, wherein the vessel is in communication with the conduit.

Example 3. The nebulizer of example 2, wherein the vessel is removably attached to the conduit.

Example 4. The nebulizer of any one of examples 2-3, wherein the vessel includes a valve configured such that medicament flows only when the cup is rotated.

Example 5. The nebulizer of any one of examples 2-4, wherein the vessel is configured such that medicament will be dispensed at a metered rate.

Example 6. The nebulizer of any one of examples 1-5, further comprising a mover for rotating the axle.

Example 7. The nebulizer of any one of examples 1-6, wherein the mover is a pair of handles, wherein at least one handle of the pair of handles is configured to rotate the axle when the handles are squeezed together by an operator.

Example 8. The nebulizer of any one of examples 1-7, wherein the cup is cone-shaped.

Example 9. The nebulizer of any one of examples 1-8, wherein the cup is made from an air permeable material.

Example 10. The nebulizer of example 9, wherein the cup is made from a mesh material.

Example 11. The nebulizer of any one of examples 1-10, wherein the drum comprises one or more ports for intake of ambient air.

Example 12. The nebulizer of any one of examples 1-11, wherein the conduit and/or the vessel includes a valve configured such that medicament flows only when the cup is rotated.

Example 13. The nebulizer of any one of examples 1-12, wherein the conduit and/or the vessel is configured such that medicament will be dispensed at a metered rate.

Example 14. The nebulizer of example 13, wherein the metered rate is adjustable.

Example 15. The nebulizer of any one of examples 1-14, wherein the axle comprises a hollow outer shaft and an inner shaft coaxially disposed within the hollow outer shaft, and wherein the fan is attached to the outer shaft and the cup is attached to the inner shaft.

Example 16. The nebulizer of any one of examples 1-15, wherein the fan is configured to rotate at a same speed as a rotational speed of the cup.

Example 17. The nebulizer of any one of examples 1-16, wherein the fan is configured to rotate at a speed different from a rotational speed of the cup.

Example 18. The nebulizer of any one of examples 1-17, wherein the conduit is configured to deposit medicament on an inner surface of the cup.

Example 19. A method of dispensing medicament, including providing a nebulizer according to any one of examples 1-18; providing medicament to the conduit of the nebulizer; and actuating the mover so as to dispense medicament.

Although the present disclosure has been described with respect to one or more particular embodiments, it will be understood that other embodiments of the present disclosure may be made without departing from the spirit and scope of the present disclosure.

1. A nebulizer for medicament, comprising:
a cup having an interior surface;
an axle attached to the cup at least at a location along a central axis of the cup such that rotation of the axle causes a rotation of the cup about the central axis;
a fan operably attached to the axle and configured to generate a flow of air adjacent to the cup;
a drum disposed around a circumference of the cup, and configured to contain the generated flow of air near the cup; and
a conduit configured to receive liquid medicament and deposit such medicament on a surface of the cup.

2. The nebulizer of claim 1, further comprising a vessel for containing a liquid medicament, wherein the vessel is in communication with the conduit.

3. The nebulizer of claim 2, wherein the vessel is removably attached to the conduit.

4. The nebulizer of claim 2, wherein the vessel includes a valve configured such that medicament flows only when the cup is rotated.

5. The nebulizer of claim 2, wherein the vessel is configured such that medicament will be dispensed at a metered rate.

6. The nebulizer of claim 1, further comprising a mover for rotating the axle.

7. The nebulizer of claim 6, wherein the mover is a pair of handles, wherein at least one handle of the pair of handles is configured to rotate the axle when the handles are squeezed together by an operator.

8. The nebulizer of claim 1, wherein the cup is cone-shaped.

9. The nebulizer of claim 1, wherein the cup is made from an air permeable material.

10. The nebulizer of claim 9, wherein the cup is made from a mesh material.

11. The nebulizer of claim 1, wherein the drum comprises one or more ports for intake of ambient air.

12. The nebulizer of claim 2, wherein the conduit and/or the vessel includes a valve configured such that medicament flows only when the cup is rotated.

13. The nebulizer of claim 2, wherein the conduit and/or the vessel is configured such that medicament will be dispensed at a metered rate.

14. The nebulizer of claim 13, wherein the metered rate is adjustable.

15. The nebulizer of claim 1, wherein the axle comprises a hollow outer shaft and an inner shaft coaxially disposed within the hollow outer shaft, and wherein the fan is attached to the outer shaft and the cup is attached to the inner shaft.

16. The nebulizer of claim 1, wherein the fan is configured to rotate at a same speed as a rotational speed of the cup.

17. The nebulizer of claim 1, wherein the fan is configured to rotate at a speed different from a rotational speed of the cup.

18. The nebulizer of claim 1, wherein the conduit is configured to deposit medicament on an inner surface of the cup.

19. A method of dispensing medicament, comprising:
providing a nebulizer according to claim 6;
providing medicament to the conduit of the nebulizer; and
actuating the mover so as to dispense medicament.
